(19) United States
(12) Patent Application Publication
(10) Pub. No.: US 2012/0165619 A1
(43) Pub. Date: Jun. 28, 2012

(54) STAND ALONE MEDICAL COMMUNICATION MODULE USED WITH A HOST DEVICE

(75) Inventors: Javaid Masoud, Shoreview, MN (US); William D. Verhoef, Andover, MN (US); Mark A. Maass, Tempe, AZ (US); Bo Zhang, Blaine, MN (US); Christopher M. Petersen, Ham Lake, MN (US); Gregory J. Haubrich, Champlin, MN (US)

(73) Assignee: Medtronic, Inc.

(21) Appl. No.: 13/336,315

(22) Filed: Dec. 23, 2011

Related U.S. Application Data
(60) Provisional application No. 61/427,338, filed on Dec. 27, 2010.

(51) Int. Cl. A61B 5/00

(52) U.S. Cl. 600/300

(57) ABSTRACT

System and method for providing medical information concerning a patient having first patient physiological data and second patient physiological data. The system has a medical device and a handheld device. The medical device is configured to be implanted in the patient and has a sensor configured to obtain the first patient physiological data and a communication module. The handheld device has a communication module, a processor and a user interface. The device communication module is configured to communicate with the medical device communication module, the device communication module being configured to transfer the first patient physiological data to the handheld device via the communication module. The processor is configured to combine the first patient physiological data and the second patient physiological data and generate feedback having a recommended course of action based, at least in part, on the first patient physiological data and the second patient physiological data.

### RELATED APPLICATION

This application claims priority from U.S. Provisional Application No. 61/427,338, filed on Dec. 27, 2010, entitled “STAND ALONE MEDICAL COMMUNICATION MODULE USED WITH A HOST DEVICE”.

### FIELD

The present invention related generally to medical communication devices and, more particularly, to devices to communicate with a medical device.

### BACKGROUND

Commercial consumer electronic devices or other so-called “off-the-shelf” electronic devices for providing computing operations and communications, both wired and wireless, are well known in the art. Devices such as personal digital assistants (“PDAs”), “smartphones” and tablet personal computers provide computing power, digital storage and user input/output functionality in what is, typically, a size and weight which is conducive to easy portability by an individual user. In addition, so-called “netbooks”, as well as notebook and laptop computers, may provide similar functionality, albeit commonly in a larger form-factor and with greater weight.

Commonly, such devices listed above incorporate a communication module or communication modules to allow the devices to communicate over various wireless communications bands. Standards such as Bluetooth, IEEE 802.11, cellular, among others known in the art, provide both protocols and designated frequencies over which communications may occur. In addition, proprietary communications schemes may be developed and fielded independently. Communication modules designed to be consistent with such commercial and proprietary standards may be incorporated into such devices to permit them to communicate wirelessly with other devices similarly designed to communicate according to the various standards.

Electrically active medical devices may similarly be configured to communicate according to commercial and proprietary communication standards. Such medical devices may be involved in communications to transmit data relating to the condition of the medical device as well as the condition of the patient with which the device is associated. In addition, the medical device may be involved with communications to receive commands from external sources pertaining to the function of the medical device, for instance to reprogram the medical device from a first configuration setting to a second configuration setting. The Medical Implant Communication Service (“MICS”) band is commonly used to communicate with an implanted medical device. The Medical Data Service (“MEDS”) is an ultra-low power medical device communication system using the 401-402 megaHertz and/or 405-406 megaHertz bands.

But while medical devices may, like commercial devices, operate according to various communication standards, the standards according to which the medical devices operate may not advantageously be the same as those to which commercial devices operate. While a commercial device may usefully communicate according to, for instance, the Bluetooth communication standard, the power requirements of Bluetooth may make using Bluetooth disadvantageous for an implantable medical device incorporating a relatively small power source. Such an implantable medical device may advantageously utilize a proprietary communication scheme over the MICS/MEDS band instead. By contrast, a smartphone, for instance, which does not commonly communicate with implantable medical devices, and which, as such, may not profitably incorporate a MICS/MEDS band receiver, may not be able to communicate with an implantable medical device.

As a result, communications with implantable medical devices have commonly incorporated proprietary, custom-designed electronic devices instead of commercial, off-the-shelf devices. Custom designed electronic devices tend to cost relatively more for design and manufacture of relatively small numbers of proprietary devices in comparison with the number of commercial devices on the market. Because of the increased cost, there may be a motivation to minimize the number of such custom-designed devices built to a relative minimum in order to save cost. This may tend to limit availability of such custom-designed electronics, reducing a utility in providing the capabilities afforded by such electronics to users other than medical professionals in a clinical setting.

### SUMMARY

It has been determined, however, that the relative proliferation of commercial devices such as smartphones, tablet computers, notebook computers and netbooks may provide an opportunity to adapt such devices with custom-designed modules to be used with existing or future commercial devices for communication with medical devices. By virtue of not being complete user-operable devices, such proprietary modules may be relatively inexpensive to manufacture and distribute. By adapting the performance of commercial devices with proprietary modules, the utility which may be provided with proprietary modules may be made available to a wide range of users beyond medical professionals in clinical settings, such as to the patients themselves or other healthcare providers, while remaining relatively cost effective.

Patients, medical professionals and medical devices may obtain greater exposure to information which may benefit the treatment of the patient through an ability to communicate between commercial devices and medical devices. By providing modules to adapt commercial electronic devices for use interfacing with and presenting information from implantable medical devices and conducting interviews and follow-ups between patients and physicians, relatively greater information and ability to interact between and among various devices and entities may be available than has been available in the past. Such information and interaction may further be made available at relatively reduced cost to health care systems than has previously been possible or realistic.

In an embodiment, a system for providing medical information concerning a patient having first patient physiological data and second patient physiological data has an implantable medical device and a handheld device. The implantable medical device is configured to be implanted in the patient and has a sensor configured to obtain the first patient physiological data and an implantable device communication module. The handheld device has a handheld device communication module, a processor and a user interface. The handheld device communication module is configured to communicate with the implantable medical device communication module, the implantable device communication module being configured to transfer the first patient physiological data to the handheld device via the handheld device communication module. The processor is configured to combine the first patient physiological data and the second patient physiological data and generate feedback based, at least in part, on the first patient physiological data and the second patient physiological data, the feedback comprising a recommended therapeutic action to be performed by at least one of the patient and a first caregiver for the patient. The user interface is configured to display the feedback.

In an embodiment, the handheld device comprises a standard consumer handheld device.

In an embodiment, the standard consumer handheld device comprises at least one of mobile telephone and a personal data assistant and personal audio (music) device.

In an embodiment, the second patient data related to the patient comprises patient data manually input to the handheld device via the user interface by at least one of the patient and a second caregiver for the patient.

In an embodiment, the patient data manually input to the handheld device comprises responses to at least one question posed to the at least one of the patient and the second caregiver for the patient by the handheld device via the user interface.

In an embodiment, the first caregiver and the second caregiver are a single person.

In an embodiment, the handheld device communication module is further configured to transmit the first patient data and the second patient data from the handheld device to a remotely located medical professional.

In an embodiment, the handheld device communication module is further configured to transmit the feedback from the handheld device to a caregiver device having a caregiver device user interface and provide the feedback to a third caregiver via the caregiver device user interface.

In an embodiment, the handheld device communication module is operationally coupled to a telemetry module, the telemetry module providing, at least in part, a telemetry connection with the implantable device communication module, wherein the implantable device communication module is configured to transmit the first patient data from the implantable medical device to the telemetry module and wherein the telemetry module is configured to send the first patient data from the telemetry module to the handheld device communication module.

In an embodiment, the telemetry module is configured to be physically coupled to the handheld device.

In an embodiment, the telemetry module is configured to wirelessly communicate with the handheld device wirelessly send at least the first patient data from the telemetry module to the handheld device.

In an embodiment, the telemetry module has a first telemetry antenna configured to communicate in a first communication band with at least one of the implantable medical device and a medical sensor device and a second telemetry antenna configured to communicate in a second communication band with at least the handheld device communication module. The medical device communication module is configured to transmit at least the first patient data to the telemetry module utilizing at least the first communication band and the telemetry module is configured to transfer the first patient physiological data to the handheld device communication module utilizing at least the second communication band.

In an embodiment, the telemetry module comprises only one telemetry antenna configured to communicate in a first communication band with at least one of the implantable medical device and a medical sensor device and to communicate in a second communication band with at least the handheld device communication module. The medical device communication module is configured to transmit at least the first patient data to the telemetry module utilizing at least the first communication band. The telemetry module is configured to transfer the first patient physiological data to the handheld device communication module utilizing at least the second communication band.

In an embodiment, a method of providing medical information concerning a patient, having an implantable medical device, on a handheld device having an user interface has the steps of transferring first patient physiological data related to the patient from the implantable medical device to the handheld device and obtaining second patient data related to the patient. Then the first patient data and the second patient data are combined in the handheld device, then feedback is generated with the handheld device based, at least in part, on the first patient data and the second patient, the feedback comprising a recommended therapeutic action to be performed by at least one of the patient and a first caregiver for the patient, and the recommended therapeutic action is provided on the user interface of the handheld device.

In an embodiment, the method further has the step of transmitting the first patient data and the second patient data from the handheld device to a remotely located medical professional.

In an embodiment, the method further has the steps of transmitting the feedback from the handheld device to a caregiver device having a user interface and providing the feedback to a third caregiver via the user interface of the caregiver device.

In an embodiment, the handheld device is locally coupled with a telemetry module, the telemetry module providing, at least in part, a telemetry connection with the implantable medical device. The transferring step has the steps of transmitting the first patient data from the implantable medical device to the telemetry module and sending the first patient data from the telemetry module to the handheld device.

In an embodiment, the telemetry module is configured to wirelessly communicate with the handheld device and the sending step comprises wirelessly transmitting at least the first patient data from the telemetry module to the handheld device.

In an embodiment, the telemetry module has a first telemetry antenna configured to communicate in a first communication band with at least one of the implantable medical device and a medical sensor device and a second telemetry antenna configured to communicate in a second communication band with at least the handheld device. The transmitting at least the first patient data to the telemetry module step utilizes at least the first communication band and the transferring step utilizes at least the second communication band.

In an embodiment, the telemetry module has only one telemetry antenna configured to communicate in a first communication band with at least one of the implantable medical device and a medical sensor device and to communicate in a second communication band with at least the handheld device. The transmitting at least the first patient data to the telemetry module step utilizes at least the first communication band and the transferring step utilizes at least the second communication band.

### FIGURES

FIG. 1 is an illustration of a network to interface between implantable medical devices in a patient, including therapy delivery devices and sensors, and outside receptors utilizing a communication module coupled to a host device;

FIG. 2 is an exemplary embodiment of a communication module coupled to a host device;

FIG. 3 illustrates an embodiment of a host device coupled to a module and configured to facilitate communications between an implantable medical device in a patient and a wider network;

FIG. 4 is a depiction of a graphical application for a host device configured to facilitate interfacing with implantable medical devices;

FIG. 5 is a diagram for conducting communications between the host device, the communication module and the implantable medical device;

FIG. 6 is a depiction of utilizing multiple host devices of varying types to communicate with multiple medical devices and to facilitate communications between and among the multiple medical devices, the host devices and the third-party devices over the Internet;

FIG. 7 is a depiction of an alternative embodiment of the communication module;

FIG. 8 is a block diagram illustrating an embodiment of the communication module which may operate without a physical connection with the host device;

FIG. 9 is a block diagram of an embodiment of a communications module using a host device for a user interface; and

FIG. 10 is a flowchart for providing medical information concerning a patient on the handheld device.

### DESCRIPTION

The entire content of U.S. Provisional Application Ser. No. 61/427,338, filed Dec. 27, 2010 is hereby incorporated by reference.

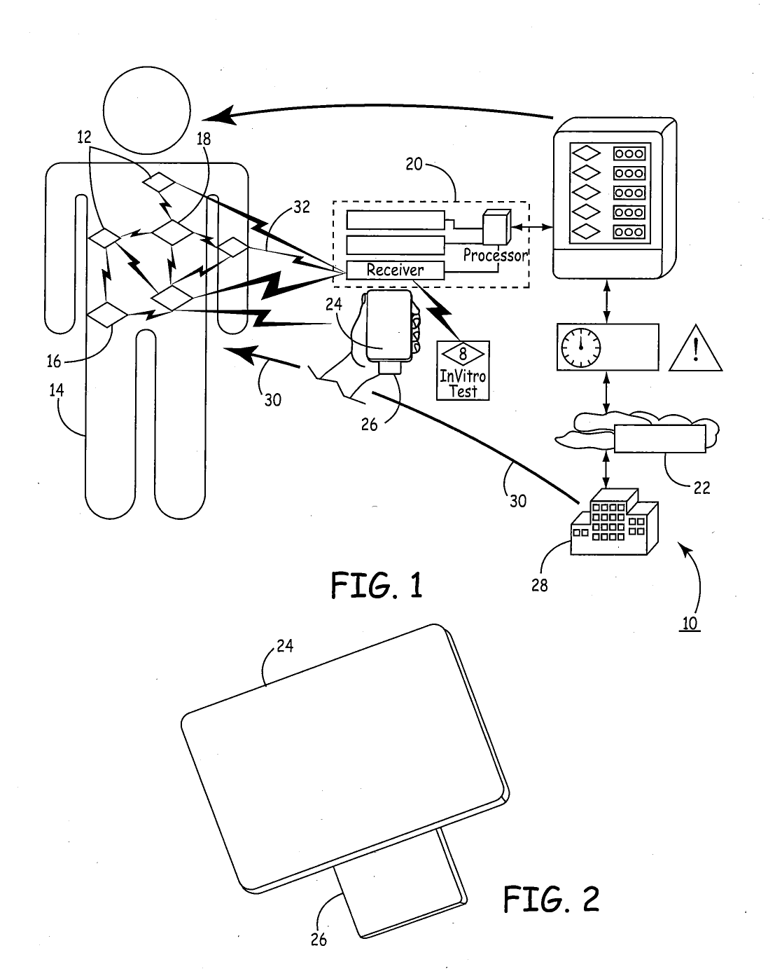

FIG. 1 is an illustration of an exemplary network 10 to interface between implantable medical devices 12 in patient 14, including therapy delivery devices 16 and sensors 18, and outside receptors 20. Information may flow from implantable medical devices 12 to external networks 22, while information and instructions may flow to implantable medical devices 12 from network 10. One device which may facilitate such information flows is standard consumer handheld electronic device 24, or host, as depicted by a smartphone, for example, an iPhone™ smartphone¹ by Apple Inc. As illustrated, host 24 is operably, locally coupled to module 26 configured to facilitate communications and between and interfacing with implantable medical devices 12 and host 24. In various embodiments described below, host device 24 is locally coupled to communication module 26 either through a physical connector or by wireless communication. Alternative embodiments of host 24 are envisioned, including, but not limited to, products by Apple Inc. such as the iPod™ digital music player², iPad™ tablet computer³ and MacBook™ computer⁴, the BlackBerry™⁵ smartphone by Research-in-Motion, Ltd., the Droid™ smartphone⁶ and the Defy™ smartphone⁷ by Motorola, Inc., the Optimus™ smartphone⁸ by LG Electronics Inc., and the Evo™ smartphone⁹ and Wildfire™ smartphone¹⁰ by HTC Corp. ¹iPhone is a trademark of Apple Inc.²iPod is a trademark of Apple Inc.³iPad is a trademark of Apple Inc.⁴MacBook is a trademark of Apple Inc.⁵BlackBerry is a trademark of Research-in-Motion, Ltd.⁶Droid is a trademark of Motorola, Inc.⁷Defy is a trademark of Motorola, Inc.⁸Optimus is a trademark of LG Electronics Inc.⁹Evo is a trademark of HTC Corp.¹⁰Wildfire is a trademark of HTC Corp.

Advantageously, the use of an off-the-shelf, commercially available consumer electronic device may provide a common and easy to use standard user interface. Such host devices 24 may incorporate a proven and robust infrastructure for the writing and dissemination of applications in support of communication module 26. Host devices 24 may incorporate a family or platform of devices which may allow for single applications which may be useful on multiple devices. In addition, such commercial devices commonly incorporate common electronic connectors, both within device platforms and families and across device platforms and manufacturers. The commercial features of host devices 24 may further be utilized in support of medical applications, such as by providing easy accessibility to email, text and other forms of electronic communication. Additionally, existing medical applications may be utilized to supplement proprietary medical applications, providing, for instance, applications for regulating patient's 14 diet, weight, blood pressure, insulin, blood glucose levels and so forth.

As illustrated, host device 24 is locally coupled to communication module 26 by way of an electronic connector (obscured). The connector may be standard for host device 24 and may be utilized by host device 24 to interface with external data sources and power supplies. In various embodiments, communication module 26 may be configured to interface with multiple different types of host devices 24, e.g., by having multiple electronic connectors or by having a common connector (for example, a USB port, that can connect to differing devices). In various alternative embodiments, each communication module 26 is configured to function with only one particular type of host device 24.

Communication module 26 may be configured to communicate wirelessly with implantable medical devices 12 in patient 14. Host device 24 and module 26 together may be configured to perform various functions relating to interfacing with medical device 12, for instance, by receiving information from one or more of implantable medical devices 12 and, in some instances, provide the received information to host device 24. Module 26 may also be configured to receive information (e.g., data or instructions) from host device 24 for transmission to implantable medical devices 12 and transmit the received information to one or more of implantable medical devices 12. Host device 24 may be variably configured to display the information received from implantable medical devices 12 and/or to forward the information received from implantable medical devices 12 to other members of network 10, illustrated or not. Host 24 may be configured to transmit the information received by way of communications methods already incorporated into host device 24. For instance, where host device 24 is a smartphone, the host device may transmit the information over a cellular network, over a WiFi network or over a physical connection such as Ethernet or modem.

Host 24 and communication module 26 may together be further variably configured to allow a user to perform functions relating to interfacing with implantable medical device 12, such as by entering instructions for transmission to implantable medical devices 12 by way of module 26. In addition, host 24 may be configured to receive instructions from a third-party device by way of host device's 24 built-in communications systems. For instance, a medical professional operating at remote site 28 may be permitted to transmit instructions 30 to host device 24 by way of the cellular system, for instance, which may then be communicated to one or more of implantable medical devices 12 by way of communication module 26.

FIG. 2 is an exemplary embodiment of communication module 26 coupled to host device 24, as illustrated a smartphone. Communication module 26 is configured with a connector which allows module 26 to be operatively connected to host device 24 according to the requirements and specifications of host device 24. As such, module 26 may be configured to be operatively connected to any similar model smartphone, in the illustrated example, interchangeably. In addition, any host device 24 with the same connection capability may be operatively connected to communication module 26.

In various embodiments, host device 24 may be configured with software, such as an application or “app” running on host device 24, to allow host device 24 to interface with communication module 26 according to the various functions described herein. Each application may correspond to one or more such function, for instance by providing a display for patient physiological data, data relating to the performance of medical device 12, and entering in programming parameters for transmittal to medical device 12, among other functions. The software may allow host device 24 to operate with module 26, display information received from implantable medical device 12 by way of module 26 and allow a user to input instructions to be transmitted to implantable medical device 12, among other functions. The software may be incorporated into module 26 and downloaded into host 24 when module 26 is plugged into host 24, or may be downloaded into host device 24 directly or remotely according to methods well known in the art.

In an embodiment, communication module 26 is configured to communicate 32 (FIG. 1) with implantable medical device 12 on the MICS/MEDS band. In one example, module 26 is approximately fifty (50) millimeters by fifty (50) millimeters and incorporates a thirty (30) pin connector. Communication module 26 incorporates one or more antennas as well as at least one processor to support communications.

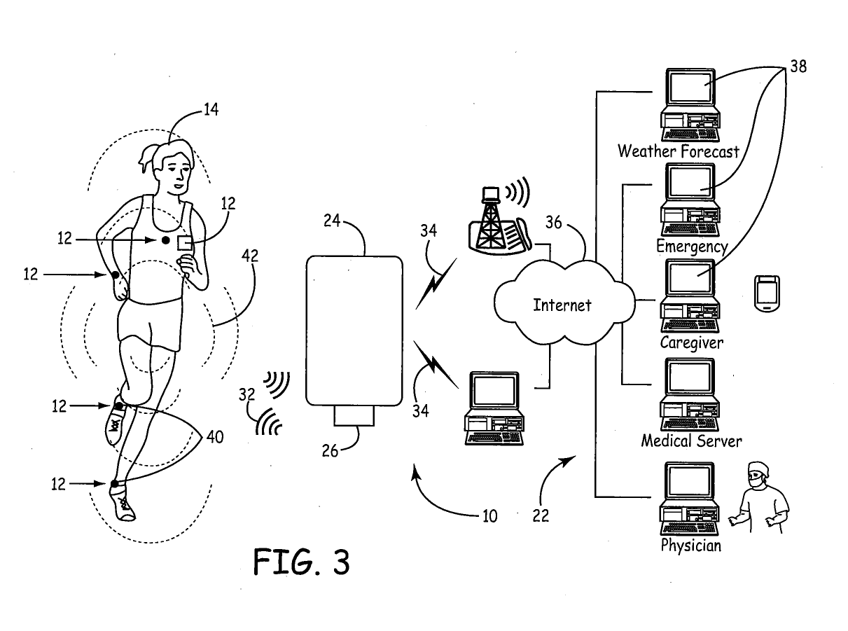

FIG. 3 illustrates an embodiment of host device 24 coupled to module 26 and configured to facilitate communications 32 between implantable medical device 12 in the patient and a wider network 22. Communication module 26 permits communication between host device 24 and implantable medical device 12 of mobile patient 14. Host device 24 permits wireless communication 34 according to various standards with the Internet 36 and thus various third-party destinations 38.

In the illustrated embodiment, host device 24 and communication module 26 may be on the person of patient 14 and transmitting as patient 14 moves around. In various embodiments, communication module 26 may be separated from implantable medical device 12 by ten (10) meters or more. However, in certain embodiments, communication module 26 may not be configured to communicate with implantable medical device 12 at ranges longer than approximately ten (10) meters and may instead have a communication range of one (1) meter or less. By contrast, host device 24 may be configured to communicate on WiFi and/or cellular bands 34, for instance, at ranges conventionally from tens of meters to multiple kilometers.

In so doing, communication module 26, coupled with host device 24, may be configured to provide global connectivity for patients with implantable medical devices 12. In various embodiments, host devices 24 which are configured to communicate over communications systems available even in relatively remote places may deliver information from patients' 14 medical devices 12 to and receive instructions from medical providers in distant places 38. In such embodiments, host devices 24 may be devices which are already possessed by patient 14 or which may be acquired at relatively modest cost. Similarly, because communication module 26 may incorporate few features and functions other than to communicate with host device 24, communication module 26 may similarly be relatively inexpensive and useable in remote areas.

In addition, the use of host devices 24 such as commercially available, “off-the-shelf” devices detailed above, may provide for patient- and physician-centric applications to support maintenance of patient's 14 implantable medical device 12 and advance patient care. Patient 14 may be provided with details of their care on host device 24 in order for patient 14 to better understand their condition and what steps patient 14 may take outside of the strict function of their implantable medical device 12 to advance their treatment. Physicians may be provided on their own devices 38, either commercial devices or purpose-built devices, information similarly related to the status of patient 14 and implantable medical device 12, and may be provided with such information conveniently and without having to directly interface with patient 14. Thus, such information may be provided conveniently and at relatively low cost. In further instances, patient 14 and the physician may use the same host device 24 with different communication modules 26 attached or the same host device 24 using the same communication module 26 with additional functionality provided to the physician (e.g., by using a password).

In particular, patient-centric applications may include monitoring and reporting to patient 14 of adverse medical events and reactions to treatment, alerts instructing patient 14 to take a particular action, and physiologic information not necessarily related to their treatment. Physiologic information may include information such as blood pressure and weight. Additional patient-centric information may include educational materials for instructing patient 14 on living with various diseases and conditions, vital signs and instruction on activities such as eating, exercise and daily health logs. Additional patient-centric applications are envisioned.

In particular, physician-centric applications may include programming capabilities for implantable medical devices 12 of patient 14, providing patient 14 with medical advice and enabling various alternative forms of communication with patient 14 and other sources. Programming capabilities may include full programming capabilities for implantable medical devices 12. Alternatively, perhaps particularly for relatively complex devices which may cause a negative impact on patient 14 in the event of a patient reaction to a new therapy, full programming of implantable medical devices 12 may be curtailed for certain, relatively more complex devices. The sharing of health and wellness information may incorporate customized data viewing capabilities, for particular devices, patients 14 and physicians, as well as generalized health information and interfaces which may be presented on other, proprietary devices.

In addition to providing patient- and physician-centric applications, communication module 26 may provide such applications while allowing implantable medical devices 12 to become or maintain relatively small size and form factor, as well as to attain or maintain relatively low power consumption. By not needing to communicate over communication bands and according to communication standards which utilize relatively large antennas and consume relatively large amounts of power, such as those found on host devices 24 listed above, implantable medical devices 12 can use relatively short-range, low-power communications schemes such as those typically and historically utilized on implantable medical devices 12 while still maintaining the benefits of long-range communications. In so doing, the relatively small form factors and low power consumption of implantable medical devices 12 may be maintained.

It is to be recognized and understood that other sensors 40 may be utilized, including and without limitation, in an embodiment, one or motion sensors (e.g., a motion sensor positioned with respect to the body core and a motion sensor positioned with respect to an extremity), one or more tilt sensors (e.g., to assist in determining either a position of the body, an angle of repose of the body or both), and one or more oxygen sensors. Any and all of sensors 40 could communicate with host device 24 by way of communication module 26. Further, any and all of sensors 40 may also communicate with each other, or some of the other sensors 40, by way of, for example, a body area network 42 using, for example the MICS/MEDS band.

In an exemplary embodiment, body area network 42 may be utilized to communicate not only with any and all of sensors 40 but also may communicate with implantable medical device 12 or multiple implantable medical devices 12. Any and all of such implantable medical devices may communicate not only with each other and with any and all of such sensors 40 but also may communicate with host device 24 through communication module 26 using, for example the MICS/MEDS band.

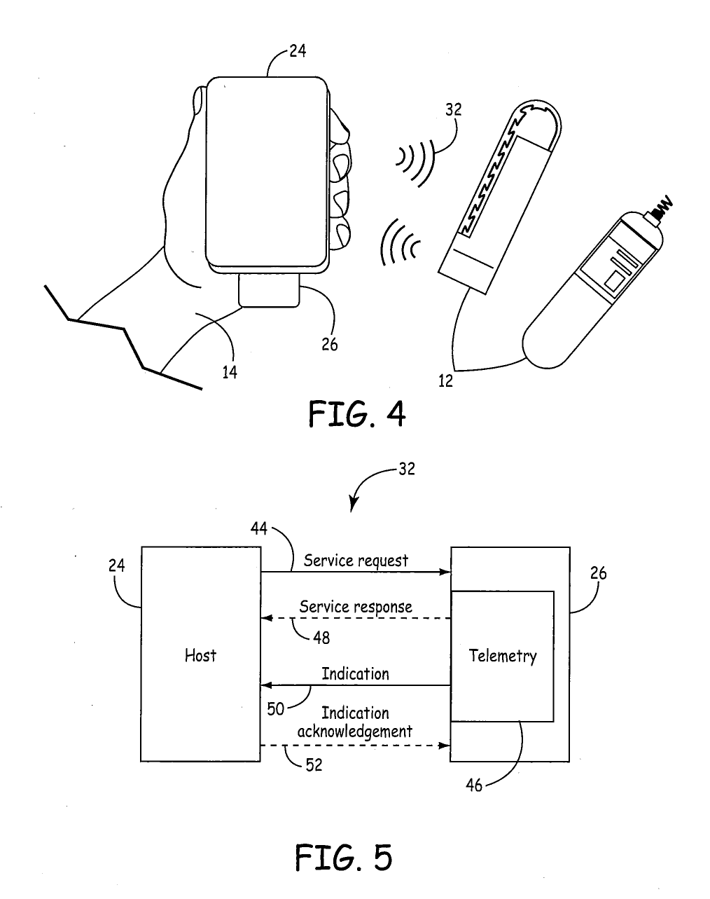

FIG. 4 is an example depiction of a graphical application for host device 24 configured to facilitate interfacing with implantable medical devices 12. As depicted, the application provides data to patient 14 relating to the conduction of a basic exercise program or “basic workout”. In such an embodiment, implantable medical device 12 may be related to providing a physiologic status of patient 14, such as blood pressure or heart rate. Because various host devices 24 incorporate different operating systems and different hardware, the various applications which are developed may be adapted for different host devices 24. For host devices 24, which incorporate a common operating system and the same or similar hardware, applications may be developed which are cross-functional.

FIG. 5 is a diagram illustrating an example series of communications 32 between host device 24, the communication module and implantable medical device 12. In particular, FIG. 5 illustrates example steps by which host device 24 initiates communication with implantable device 12 by making a service request 44 to telemetry module 46 of communication module 26 and receiving service response 48. Services which may be requested include, but are not necessarily limited to, a command to initialize, discover the presence of medical device 12, open communications, obtain data and close communications. FIG. 5 further illustrates the initiation of communication or, alternatively, the response to the service request by the communication module by transmitting indication signal 50. Such functions may be implemented in firmware on communication module 26 and may be acknowledged with indication acknowledgement 52.

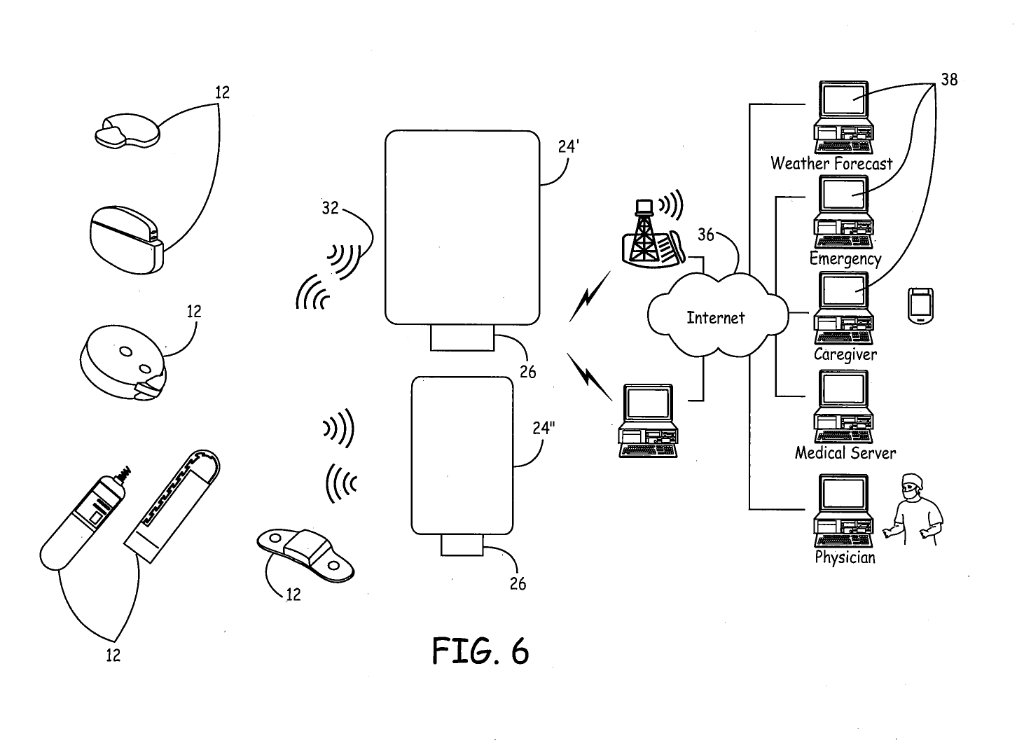

FIG. 6 is a depiction of utilizing multiple host devices 24 of varying types to communicate 32 with multiple medical devices 12 and to facilitate communications between and among multiple medical devices 12, host devices 24 and the third-party devices 38 over the Internet 36. As illustrated, both a tablet computer host device 24′ and a smartphone host device 24″, in an embodiment an iPad™ tablet computer and an iPhone™ smartphone, respectively, are configured to communicate 32 with various medical devices 12, both implantable and non-implantable. The presence of multiple medical devices 12 and multiple host devices 24 need not interfere with the ability of various medical devices 12 and host devices 24 to communicate with one another.

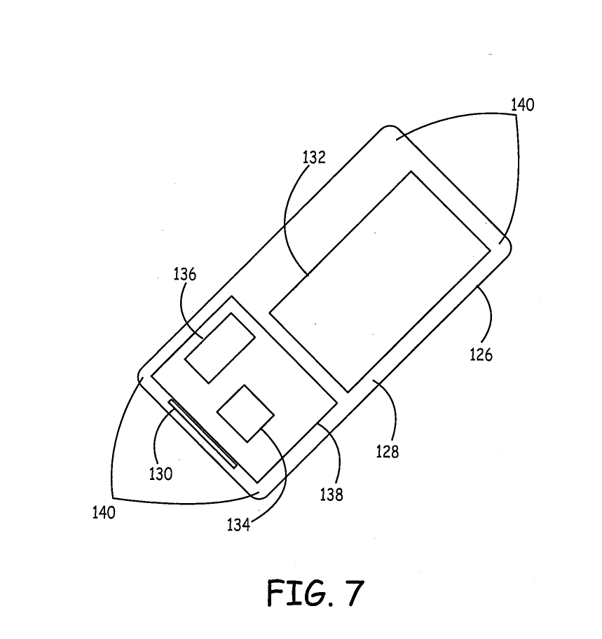

FIG. 7 is a depiction of an alternative embodiment of communication module 126. Rather than being a plug-in-style module 26 as shown, for instance, in FIG. 2, the communication module of FIG. 7 is incorporated in a casing or “skin” 128 to which host 24 device may be positioned, e.g., by “skin” 128 partially enveloping host device 24. As illustrated, casing 128 incorporates connector or data cord 130 to physically interface with host device 24. Electronics, including power source 132, processor 134, memory 136, motherboard 138 and antenna 140 are incorporated into the casing. Such components may be incorporated so as to be relatively flush with casing 128, thereby reducing the extent to which casing 128 increases the form factor of host device 24 relative, for instance, to the dongle implementation of communication module 26.

It is to be recognized and understood that, while the embodiments described above depict communication module 26, 126 configured to make a physical connection with host device 24, alternative embodiments of communication module 26 may be implemented. In particular, communication module 26 may be configured to operate without a physical connection to host device 24. In such embodiments, communication module 26 may have a power source such as battery 132 independent of host device 24 and may communicate with host device 24 according to various communication schemes detailed above with respect to communication between communication module 26 and medical device 12. Such communication schemes may include, but are not necessarily limited to, cellular, Bluetooth and WiFi. In such embodiments, host device 24 may be configured to maintain wireless communications with third party devices 38 according communication schemes described above, including, in various embodiments, the same scheme utilized for communications between communication module 26 and host device 24, without inhibiting communications between host device 24 and communication module 26 and the third party devices 38.

Providing patients 14 and physicians with relatively greater access to information and control of medical devices 12 may be beneficial in terms of the ability of patient 14 to understand and improve their own condition and the ability of a physician to treat patient 14. However, the proliferation of information and control may have side effects which may be mitigated. In particular, if a third party were to be able to access host device 24 with a coupled communication module 26, the third party may be able access personal and sensitive information about patient 14 and may, in certain circumstances, be able to impact the performance of patient's 14 device 12.

In various embodiments described above, communication module 24 is plugged into host device 24 via connector 130 and receives power from host device 24 for some or all of the power requirements for communication module 26. It is noted that host device 24 typically has power constraints and limitations of its own; whether internally powered with a battery or via an external power source, host device 24 may not be able to provide as much power as devices peripheral to host device 24 may maximally consume. Further, connector 130 with which communication module 26 connects with host device 24 may have current and/or power limits set or imposed by the manufacturer, provider, operator or user of host device 24. In an embodiment, although still connected to and receiving power from host device 24, communication module 26 incorporates its own power source 132, e.g., from a battery, whether primary or rechargeable, which may at least partially power communication module 26, thus limiting or reducing the amount of power received or required from host device 24.

In certain circumstance, communication module 26 may receive all or substantially all of the power required for some or most configurations or operations of communication module 26 or during some or most of the time that communication module 26 is operative. However, in certain configurations or during certain operations or at certain times, communication module 26 may need additional power, supplementary power or substitute power from host device 24. In an exemplary embodiment, communication module 26 may need to receive power from host device 24 or may need to receive additional power from host device 24 when power source 132 is depleted or approaches depletion or is in any way reduced from its optimal or normal performance. In this way, host device 24 may serve as a backup or supplementary power source for communication module 26 without communication module 26 being an undue burden on the operation of host device 24.

In various embodiments, communication module 26 is configured to operate both directly coupled to host device 24 and without a physical connection to host device 24. In an alternative embodiment, communication module 26 may be configured to operate without a physical connection to host device 24 and without any ability to physically connect with host device 24, i.e., does not incorporate connector 130. In various such embodiments, communication module 26 may be configured to communicate wirelessly with host device 24, in various embodiments via antenna 140. In one such embodiment, communication module 26 may be configured to physically connect with host device 24 or transfer data via a wireless scheme to and from host device 24.

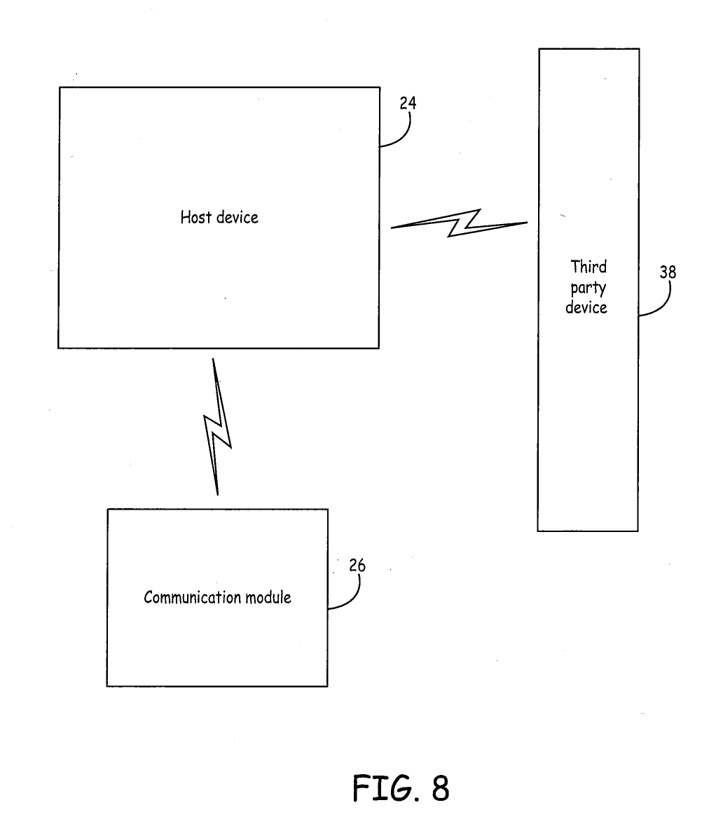

FIG. 8 is an embodiment of communication module 26 configured to communicate with host device 24 according to communication protocols and schemes which are incorporated in host device 24 by the manufacturer of host device 24. In an embodiment, host device 24 and communication module 26 communicate according to the Bluetooth protocol. In an alternative embodiment, host device 24 and communication module 26 communicate according to WiFi, cellular or various additional wireless protocols. In various embodiments, host device 24 and communication module 26 are configured to communicate according to one such protocol. In an alternative embodiment, host device 24 and communication module 26 are configured to communicate according to multiple such protocols. In various embodiments, host device 24 is also configured to interface with third party device 142 via a wireless scheme. In various embodiments, host device 24 communicates with third party device 142 according either to the same communication scheme used with communication module 26 or with a different communication scheme. In various embodiments, host device 24 is configured to communicate with third party device 142 according to at least one of the schemes detailed above.

In various embodiments, antenna 140 may be configured to facilitate communications with both medical device 12 and host device 24. In one such embodiment, antenna 140 is configured to facilitate communications with medical device 12 on the MICS band and with host device 24 via Bluetooth. Advantageously, antenna 140 may be capable of communicating over the MICS band and Bluetooth with only a need for conventional tuning and trimming circuitry. In alternative embodiments, antenna 140 is configured to communicate over different bands, in an embodiment with a proprietary communication scheme for communication with the medical device and WiFi for communication with host device 24 which advantageously may be accomplished with conventional tuning and trimming circuitry.

In an alternative embodiment, communication module 26 incorporates a pair of antennas 140, one for communication with medical device 12 and the other for communication with host device 24. While such an embodiment may utilize relatively more volume and be relatively more costly than the embodiments with a single antenna 140, the module may utilize communications bands and schemes which may be inefficient when combined on a single antenna 140. In such an embodiment, antennas 140 may utilize a single transceiver. In an alternative embodiment, antennas 140 may be positioned on separate ground planes and utilize separate transceivers.

Embodiments of communication module 26 which do not incorporate connector 130 may necessarily incorporate internal power source 132. In various embodiments, internal power source 132 is a battery. In an embodiment, power source 132 is rechargeable. In alternative embodiments, power source 132 is a non-rechargeable battery, which, in an embodiment, may be commercially available and replaceable battery cells. Alternatively, the battery may not be replaceable, with the communication module 26 being returned for depot refurbishment or scrapped when the battery depletes. Further alternatively, communication module 26 may be coupled to an external power source, such as a conventional wall outlet. In embodiments with a rechargeable power source 132, communication module 26 may be associated with a recharging unit which may itself draw power from a wall outlet or other source.

Alternatively, in embodiments where the communication module incorporates internal power source 132 and connector 130 for coupling directly to host device 24, the power supplied by host device 24 may supplant the power supplied by power source 132. Further alternatively, power source 132 may be recharged from host device 24. In additional embodiments, communication module 26 may draw power from internal power source 132 when coupled to host device 24 in order to, extend the useful life of host device 24.

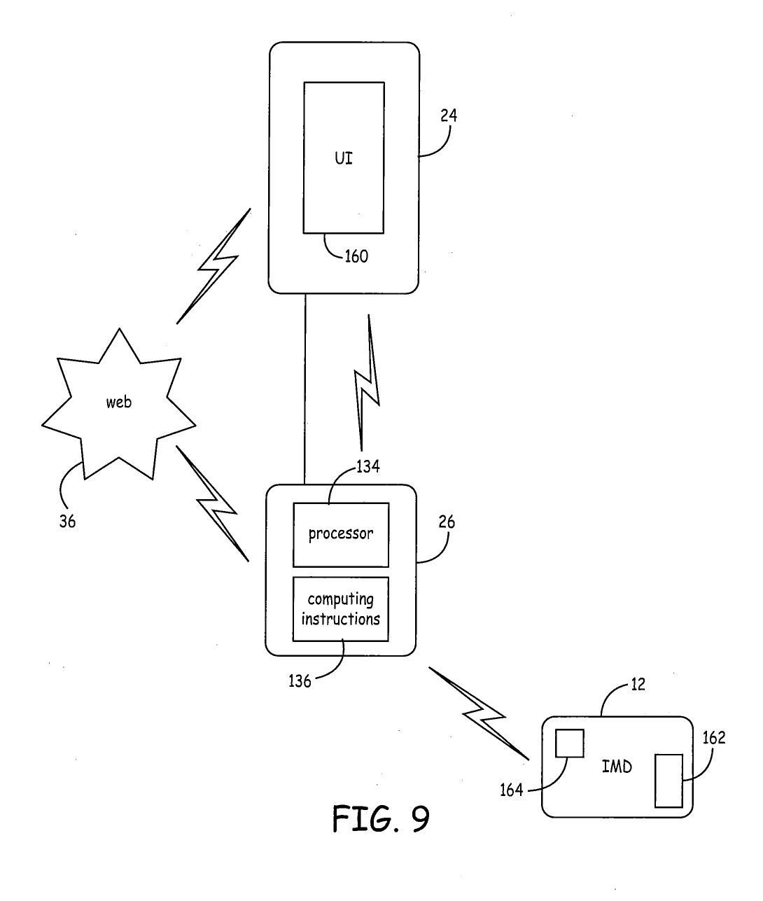

FIG. 9 is a block diagram of host device 24 having user interface 160 and configured to communicate with communication module 26 according to wired and/or wireless schemes described in detail above. In embodiments in which host device 24 and communication module 26 are coupled together, they may be deemed a single handheld device. As detailed above, communication module incorporates processor 134 and memory 136 storing computing instructions. As noted above, host device 24 may also incorporate processors and memory modules. Host device 24 is further configured to communicate wirelessly with the Internet 36 while communication device is configured to communicate wirelessly with implantable medical device 12.

In the embodiment of FIG. 9, communication module 26 is configured to be readily portable with a patient. In certain embodiments, communication module 26 is configured as a conventional object that the patient may carry on their person most of the time, such as a key fob. In various such embodiments, communication module 26 is configured to not be coupled directly with host device 24, as detailed above. Alternatively, communication module 26 is configured with a connector to be coupled directly to host device 24, as described above. In such embodiments, communication module 26 may incorporate a member to cover connector 130 in order to prevent damage to connector 130.

Implantable medical device 12 may incorporate sensors 162 for detecting a patient physiological data such as heart rate, blood pressure and the like. Such sensors are well known in the art. Implantable medical device further incorporates implantable device communication module 164 configured to communicate with communication module 26. Additional patient physiological data may be obtained by way of user interface 160, in an embodiment using patient-centric or physician-centric applications described in detail above. Such patient-centric or physician-centric applications may obtain information including patient physiological data relating to weight or blood pressure. Further, patient physiological data may be obtained by way of a peripheral device configured to communicate with at least one of host device 24 and communication module 26, such as patient weight from a scale configured with a wireless transmitter configured to be received by host device 24 or communication module 26.

The processor and other relevant electronics of host device 24 and/or processor 134 of communication module 26 may be utilized to pose requests for information by incorporating questions such as “how are you feeling” and “did you take your medicine”. Responses to such requests may be, in various embodiments, either a binary yes or no answer or a selected number indicative of a comparative level. In alternative embodiments, the patient responses may be based not on concrete numerical or binary responses, but may be based on colloquial responses, such as “fine” or “not well”. Such responses may be utilized by artificial intelligence applications known in the art to provide an indication of a patient condition useful for medical diagnosis.

On the basis of the various patient physiological information obtained, a processor of host device 24 or processor 134 of communication module 26 may generate feedback for displaying on user interface 160. Such feedback may provide patient-centric or physician-centric information as described in detail above. Such patient-centric or physician-centric information may incorporate a report of the patient's condition and may include a recommended therapeutic action, such as “take your medicine now” or instructions to reprogram medical device 12 with new parameters and the like. The information may further be transmitted to a remote device 38 for viewing and implementation by a caregiver or medical professional. Patient-centric and physician-centric applications may be utilized in conjunction with the patient-centric or physician-centric information described above to display such information and allow a user to manipulate or otherwise interact with such information via user interface 160 or a user interface of a peripheral or remote device.

In such embodiments configured to be carried on the patient's person, communication module 26 may be configured to interface only with a single, pre-identified host device 24. Alternatively, communication module 26 may be configured allow communications with any host device 24 within a communication range of communication module 26. In certain such embodiments, the patient or other user of system 10 may access communication module 26 with host device 24 according to a secure system to prevent or reduce a likelihood of tampering. In various such embodiments, host devices 24 to which communication module 26 may be configured to be connected when in range include a smartphone, a personal computer and a television configured to display messages and various other devices as detailed above.

In various embodiments, communication module 26 incorporates a user interface. In some embodiments, the user interface is relatively limited, incorporating variably lights, speakers and vibration units configured to provide alerts to the patient or to convey simple information. For instance, a light may prompt the user to recharge the module while an auditory alarm and vibration may notify the patient that the patient's medical device has detected a condition for which medical treatment is required or otherwise recommended.

On this basis, communication module 26 may operate entirely outside of the scope of host device 24 and still provide the patient with useful, even life-saving information regarding the patient's condition. Nevertheless, it may remain advantageous to operate the module in the context of host device 24 in order to provide the patient with greater information than may be presented on communication module 26, and to permit the transmission of information from medical device 12 to the patient's physician and other medical professionals as detailed above.

As illustrated, instead of relying on host device 24 for computational and/or processing to accomplish medical device 12, such as one or more implantable medical devices 12, functions or applications as identified above, communication module 26 is configured with processor 134 and memory 136 to store computing instructions to handle some or all of any such computational and/or processing tasks associated with accomplishing a medical device 12 related activity. That is, instead of communication module 26 functioning merely as a relay with communication module 26 interfacing with host device 24 to run an application or applications, communication module 26 could function more broadly with the ability and function to perform at least some and perhaps most or all of the computational and/or processing power needed without relying on host device 24 to provide such a function. In an embodiment, communication module 26 provides some or all of the communication capability described above with respect to other embodiments and, in addition, would provide the ability to run device specific applications or other medical applications directly in communication module 26. In an embodiment, host device 24 would provide at least some or all of the user interface for the user via user interface 160. In such embodiments, communication module 26 communicates with host device 24 via connector 130 or by certain of the wireless communication techniques described above.

In various embodiments, because host device 24 is a conventional, off-the-shelf consumer device, user interface 160 is readily useable by a wide variety of users. Conventionally, user interface 160 is one or more of a display screen, touch screen and keyboard. Host device may not need to be configured specifically for the medical application or reconfigured to perform a task with which it is not usually associated if communication module 26 is configured to process the information.

As illustrated, communication module 26 is configured to communicate with the Internet 36 to communicate with remote locations. However, various communication modules 26 would not incorporate a capacity to communicate directly with the Internet 36. In various embodiments, communication module 26 may be packaged with and/or shipped with a particular medical device 12 from a manufacturer, wholesaler, hospital or other vendor to a depot or use destination. When medical device 12 and communication module 26 are to be utilized, communication module 26 may be partnered with host device 24. As host device 24 is partnered with communication module 26, the function or operation of host device 24 may be altered from, for example, a general purpose device, to a specific medical appliance through the use of an application or applications run on or downloaded to either host device 24 or communication module 26. In an example, communication module 26 tailored for a particular medical device 12 or a particular medical function, either during manufacture or later by downloading or configuration, may be paired with medical device 12. The paired communication module 26 and medical device 12 could then be utilized with a variety of host devices 24 that the user already has, is familiar with or prefers and with which the user is already familiar with its user interface 160.

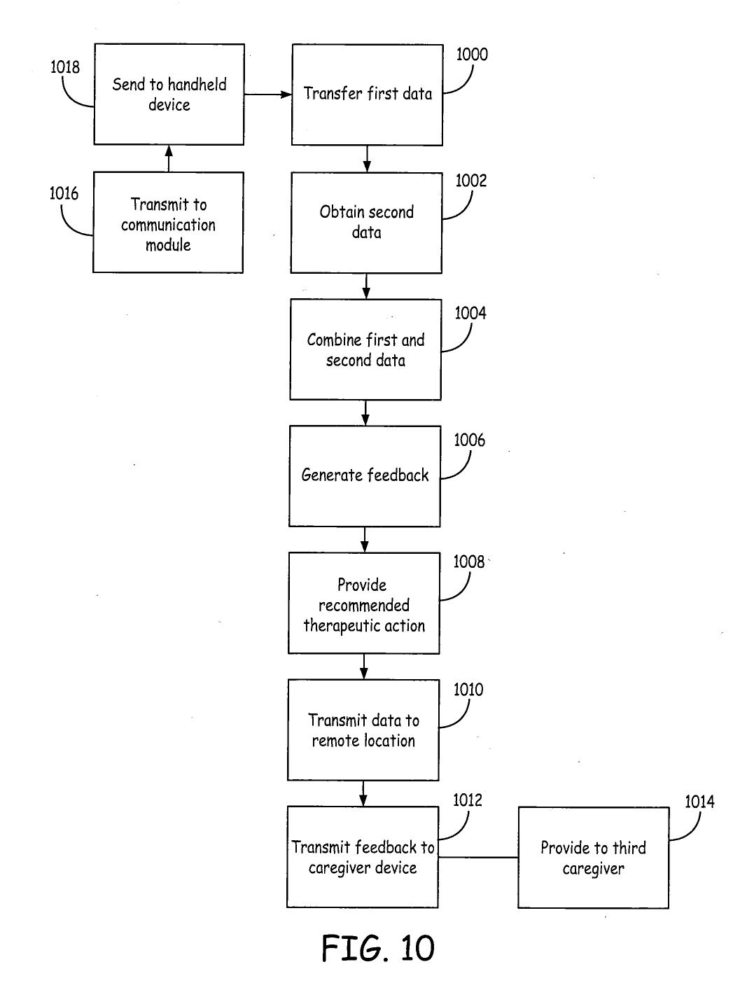

FIG. 10 is a flowchart for providing medical information concerning a patient, having an implantable medical device 12, on a handheld device, i.e., host device 24 having user interface 160 and, in various embodiments, incorporating communication module 26. First patient physiological data is transferred (1000) from medical device 12 to handheld device 24. Such first patient physiological data may include data detected by medical device 12, such as heart rate, blood pressure and the like. Second patient physiological data is obtained (1002). Then, the first patient data and the second patient physiological data is combined (1004) in handheld device 24. Feedback is generated (1006) with handheld device 24 based, at least in part, on the first patient physiological data and the second patient physiological data. The feedback includes a recommended therapeutic action to be performed, variably by the patient or a caregiver. The recommended therapeutic action is provided (1008) via user interface 160.

In certain embodiments, the first patient physiological data and the second patient physiological data is transmitted (1010) to a remotely located medical professional. The feedback may be transmitted (1012) from handheld device 24 to a caregiver device, in an embodiment another host device 24, and provided (1014) to a third caregiver, such as a medical professional. The first patient physiological data may be transmitted (1016) from medical device 12 to telemetry module 46 of communication module 26, and from there sent (1018) to handheld device 24.

Thus, embodiments of the invention are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A system for providing medical information concerning a patient having first patient physiological data and second patient physiological data, comprising:
an implantable medical device configured to be implanted in said patient and having:
a sensor configured to obtain said first patient physiological data; and
an implantable device communication module; and
a handheld device, having:
a handheld device communication module configured to communicate with said implantable medical device communication module, said implantable device communication module being configured to transfer said first patient physiological data to said handheld device via said handheld device communication module;
a processor configured to combine said first patient physiological data and said second patient physiological data and generate feedback based, at least in part, on said first patient physiological data and said second patient physiological data, said feedback comprising a recommended therapeutic action to be performed by at least one of said patient and a first caregiver for said patient; and
a user interface configured to display said feedback.

2. The system of claim 1 wherein said handheld device comprises a standard consumer handheld device.

3. The system of claim 2 wherein said standard consumer handheld device comprises at least one of mobile telephone and a personal data assistant and personal audio (music) device.

4. The system of claim 1 wherein said second patient data related to said patient comprises patient data manually input to said handheld device via said user interface by at least one of said patient and a second caregiver for said patient.

5. The system of claim 3 wherein said patient data manually input to said handheld device comprises responses to at least one question posed to said at least one of said patient and said second caregiver for said patient by said handheld device via said user interface.

6. The system of claim 4 wherein said first caregiver and said second caregiver are a single person.

7. The system of claim 1 wherein said handheld device communication module is further configured to transmit said first patient data and said second patient data from said handheld device to a remotely located medical professional.

8. The system of claim 1 wherein said handheld device communication module is further configured to:
transmit said feedback from said handheld device to a caregiver device having a caregiver device user interface; and
provide said feedback to a third caregiver via said caregiver device user interface.

9. The system of claim 1 wherein said handheld device communication module is operationally coupled to a telemetry module, said telemetry module providing, at least in part, a telemetry connection with said implantable device communication module, wherein said implantable device communication module is configured to transmit said first patient data from said implantable medical device to said telemetry module and wherein said telemetry module is configured to send said first patient data from said telemetry module to said handheld device communication module.

10. The system of claim 9 wherein said telemetry module is configured to be physically coupled to said handheld device.

11. The system of claim 9 wherein:
said telemetry module is configured to wirelessly communicate with said handheld device: and
wherein said telemetry module is configured to wirelessly send at least said first patient data from said telemetry module to said handheld device.

12. The system of claim 11 wherein:
said telemetry module comprises:
a first telemetry antenna configured to communicate in a first communication band with at least one of said implantable medical device and a medical sensor device; and
a second telemetry antenna configured to communicate in a second communication band with at least said handheld device communication module;
wherein said medical device communication module is configured to transmit at least said first patient data to said telemetry module utilizing at least said first communication band; and
wherein said telemetry module is configured to transfer said first patient physiological data to said handheld device communication module utilizing at least said second communication band.

13. The system of claim 11 wherein:
said telemetry module comprises only one telemetry antenna configured to communicate in a first communication band with at least one of said implantable medical device and a medical sensor device and to communicate in a second communication band with at least said handheld device communication module;
wherein said medical device communication module is configured to transmit at least said first patient data to said telemetry module utilizing at least said first communication band; and
wherein said telemetry module is configured to transfer said first patient physiological data to said handheld device communication module utilizing at least said second communication band.

14. A method of providing medical information concerning a patient, having an implantable medical device, on a handheld device having an user interface, comprising the steps of:
transferring first patient physiological data related to said patient from said implantable medical device to said handheld device;
obtaining second patient physiological data related to said patient; then
combining said first patient physiological data and said second patient physiological data in said handheld device; then
generating feedback with said handheld device based, at least in part, on said first patient physiological data and said second patient physiological data, said feedback comprising a recommended therapeutic action to be performed by at least one of said patient and a first caregiver for said patient; and
providing said recommended therapeutic action on said user interface of said handheld device.

15. The method of claim 14 wherein said handheld device comprises a standard consumer handheld device.

16. The method of claim 15 wherein said standard consumer handheld device comprises at least one of mobile telephone and a personal data assistant and personal audio (music) device.

17. The method of claim 14 wherein said second patient physiological data related to said patient comprises patient physiological data manually input to said handheld device by at least one of said patient and a second caregiver for said patient.

18. The method of claim 17 wherein said second patient physiological data manually input to said handheld device comprises responses to at least one question posed to said at least one of said patient and said second caregiver for said patient by said handheld device via said user interface.

19. The method of claim 18 wherein said first caregiver and said second caregiver are a single person.

20. The method of claim 14 further comprising the step of transmitting said first patient physiological data and said second patient physiological data from said handheld device to a remotely located medical professional.

21. The method of claim 14 further comprising the steps of:
transmitting said feedback from said handheld device to a caregiver device having a user interface; and
providing said feedback to a third caregiver via said user interface of said caregiver device.

22. The method of claim 14 wherein said handheld device is locally coupled with a telemetry module, said telemetry module providing, at least in part, a telemetry connection with said implantable medical device, and wherein said transferring step comprises the steps of:
transmitting said first patient physiological data from said implantable medical device to said telemetry module; and
sending said first patient physiological data from said telemetry module to said handheld device.

23. The method of claim 22 wherein said telemetry module is configured to be physically coupled to said handheld device.

24. The method of claim 22 wherein:
said telemetry module is configured to wirelessly communicate with said handheld device: and
wherein said sending step comprises wirelessly transmitting at least said first patient physiological data from said telemetry module to said handheld device.

25. The method of claim 24 wherein:
said telemetry module comprises a first telemetry antenna configured to communicate in a first communication band with at least one of said implantable medical device and a medical sensor device and a second telemetry antenna configured to communicate in a second communication band with at least said handheld device;
said transmitting at least said first patient physiological data to said telemetry module step utilizes at least said first communication band; and
said transferring step utilizes at least said second communication band.

26. The method of claim 24 wherein:
said telemetry module comprises only one telemetry antenna configured to communicate in a first communication band with at least one of said implantable medical device and a medical sensor device and to communicate in a second communication band with at least said handheld device;
said transmitting at least said first patient physiological data to said telemetry module step utilizes at least said first communication band; and
said transferring step utilizes at least said second communication band.
